(19) United States
(12) Patent Application Publication
(10) Pub. No.: US 2024/0207597 A1
(43) Pub. Date: Jun. 27, 2024

(54) VENTED MEDICAL FLUID SUPPLY LINE CAP, ASSEMBLY AND METHOD THEREFOR

(71) Applicants: Baxter International Inc., Deerfield, IL (US); Baxter Healthcare SA, Glattpark (Opfikon) (CH)

(72) Inventors: Sumana Roychowdhury, Bangalore (IN); Pushpa Yadav, District-Ghazipur UP (IN); Vandana Sakhare, Wagholi (IN)

(21) Appl. No.: 18/392,799

(22) Filed: Dec. 21, 2023

(30) Foreign Application Priority Data
Dec. 26, 2022 (IN) 202241075522

(51) Int. Cl. A61M 39/20; A61L 2/07; A61M 39/00

(52) U.S. Cl. CPC A61M 39/20; A61L 2/07; A61M 2039/0009; A61M 2205/7536

(57) ABSTRACT

A medical fluid container tubing assembly includes a medical fluid container, a luer connector including a luer port, a tube extending from the medical fluid container and terminating at the luer connector, and a cap fitted onto the luer connector. The cap includes a port sized to provide an interference fit with the luer port of the luer connector. The cap further includes a hydrophobic filter positioned and arranged to allow air to aseptically enter the tube to equalize pressure inside and outside of the tube. The cap may include an inner port including an inner surface tapered for forming an interference fit with an outer surface of a male luer port of a luer connector, an outer shroud sized to fit around an outer shroud of the luer connector, a lumen extending from the port to an opening, and a hydrophobic filter covering an opening formed by the lumen.

### PRIORITY CLAIM

This application claims priority to and the benefit as a non-provisional application of Indian Provisional Patent Application No. 202241075522, filed Dec. 26, 2022, the entire contents of which are hereby incorporated by reference and relied upon.

### BACKGROUND

The present disclosure relates generally to medical fluid treatments, and in particular to medical fluid treatments using premade or bagged medical fluid.

### BACKGROUND

Due to various causes, a person's renal system can fail. Renal failure produces several physiological derangements. It is no longer possible to balance water and minerals or to excrete daily metabolic load. Toxic end products of metabolism, such as, urea, creatinine, uric acid, and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins, and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

Any of the above treatment modalities may operate with premade, e.g., bagged, solutions. Bagged solutions are typical for any type of PD (CAPD or APD). A bagged solution may also be used for HD, especially HHD (see for example U.S. Pat. No. 8,029,454 assigned to the assignee of the present application). Continuous renal replacement therapy (“CRRT”) is an acute form of HD, HF or HDF and typically uses bagged dialysis fluids.

Premade, e.g., bagged, solutions for any of the above modalities are typically sterilized after filling and then capped to maintain the medical fluid in a sterilized condition until use. There are different ways to access the sterilized solutions at the time of use. One way is to spike the connector at the time of use, establishing medical fluid flow between the bag and a use point, such as a patient or disposable cassette. Another way, which is very common with PD is to use a breakable frangible. The patient or caregiver bends and snaps open the breakable frangible to thereafter allow fluid flow. A further way is to make a luer connection, which is a known connection that involves threading mating luer connectors together to form a liquid-tight seal between the connectors.

Regardless of the type of connection made, a bag tube is typically provided, which extends from the bag to the connector, such that there is play or room for the operator to grasp and manipulate the connector while making the fluid-tight connection to a mating connector. It has been found that steam sterilization of the bagged solution causes the bag tube to collapse. Here, because the steam outside the bag tube is at a higher pressure than the air inside the short tube, air is pushed from the tube into the bag, causing the tube to collapse. If the tube does not reopen after steam sterilization, then the bag tube is occluded at the time the solution bag is connected for use.

There is accordingly a need for an improved apparatus and associated methodology for the steam sterilization of bagged dialysis treatment solutions.

### SUMMARY

The present disclosure involves the use of a vented medical fluid supply line cap for use with a solution container or bag that is operable with any type of dialysis treatment including any type of peritoneal dialysis (“PD”) treatment, hemodialysis (“HD”) treatment, hemofiltration (“HF”) treatment, hemodiafiltration (“HDF”) treatment, or continuous renal replacement therapy (“CRRT”) treatment. It should be appreciated that the vented medical fluid supply line cap may be used in any type of medical treatment having a bagged or otherwise stored medical fluid, which needs to be opened aseptically for use, and which is steam sterilized. The vented medical fluid supply line cap may therefore be used additionally with any type of bagged medical infusion or intravenous fluid, saline, lactated ringers, etc.

The vented medical fluid supply line cap is in one embodiment configured to cap a luer connector. It should be appreciated however that the medical fluid supply line cap does not have to cap a luer connector and may instead cap a different type of connector. In any case, the cap caps a short line or tube, which may also be called a pigtail, wherein the short line or tube extends from a medical fluid supply container, e.g., a flexible bag. The flexible bag may be a single chamber bag holding a fully mixed medical fluid or be a multi-chamber bag having one or more peel seals separating medical fluid components that need to be isolated until the time of use.

The vented medical fluid supply line cap is in one embodiment formed, e.g., molded as one piece, from a polymer, such as, polyetherimide (“PEI”), polyethersulfone (“PES”), polyamide/nylon (“PA”), acrylonitrile butadiene styrene (“ABS”), polycarbonate (“PC”) polyvinylchloride (“PVC”), nylon, polyether ether ketone (“PEEK”) and/or a thermoplastic elastomer, such as one marketed under the tradename Hytrel®. The medical fluid container or bag and the bag tube (which may be a short tube or pigtail) may be made of PVC or other suitable medically safe material.

Where the vented medical fluid supply line cap is configured to cap a luer connector, the cap is one embodiment configured to have female luer features, while the luer connector is a male luer connector. In alternative embodiments, the cap may be provided with male luer features, while the luer connector is a female luer connector. Where the vented medical fluid supply line cap is configured as having female luer features, the cap includes a body having an inner port and an outer shroud. An inner surface of the inner port in one embodiment includes a female luer taper that forms an interference fit with an outer surface of an inner male luer port of the mating male luer connector. An outer surface of the inner port is in one embodiment provided with a plurality of ribs, e.g., extending longitudinally along the outer wall. The ribs form a secondary interference fit with female threads provided on an inner surface of an outer shroud of the mating male luer connector. The female threads threadingly connect to male threads of a female luer connector to establish medical fluid flow when the vented medical fluid supply line cap is removed from the male luer connector.

An inner surface of the outer shroud is sized in one embodiment to provide a slight amount of clearance with an outer surface of the outer shroud of the mating male luer connector. The clearance allows the interference fit between the inner surface of the inner port of the cap and the outer surface of the inner male luer port of the mating male luer connector to be the primary interference fit that prevents medical fluid from leaking out of the vented medical fluid supply line cap of the present disclosure.

In one embodiment, the vented medical fluid supply line cap is translated onto and off of the mating male luer connector with the interference fit holding the cap onto the male luer connector. In an alternative embodiment, the plurality of ribs provided on the outer surface of the inner port of the cap are replaced with male luer threads that threadingly connect with the female luer threads provided on the inner surface of the outer shroud of the mating male luer connector. Here, the cap threads onto and off of the mating male luer connector in the same manner as the female luer connector used to establish medical fluid flow. In either situation (translated or threaded), an outer surface of the outer shroud of the cap is formed with a plurality of ribs, e.g., longitudinally extending ribs, which aid the user in translating or threading the cap onto or off of the mating male luer connector.

In an embodiment, the lumen formed by the inner surface of the inner port extends through the body to and through a circular distal end portion of the body. An enlarged diameter cavity is provided at the distal end of the body in one. The cavity provides a location to attach a vent, such as a hydrophobic membrane filter, within the cavity, such that the opening through the body of the cap is covered. The hydrophobic filter or vent allows air but not medical fluid to flow through the filter or vent, into or out of the body of the cap. Also, air flowing into the body of the cap is aseptically filtered via the hydrophobic filter or vent. In an embodiment, the hydrophobic filter or vent includes a 0.2 micron polytetrafluoroethylene (“PTFE”) hydrophobic membrane having a polyester substrate. The hydrophobic filter or vent may be sealed around its outer diameter to the cavity of the body via heat sealing, ultrasonic sealing or solvent bonding.

When a medical fluid container or bag filled with medical fluid and having a bag tube (e.g., short tube or pigtail) extending from the container, which is capped by the vented cap of the present disclosure is steam sterilized, the hydrophobic filter or vent prevents the short tube or pigtail from collapsing. Steam sterilization typically involves many medical fluid containers or bags being placed within an autoclave and steam sterilized at the same time. The external environment around the medical fluid container or bag is filled with steam, raising the pressure in the autoclave to above ambient pressure (e.g., 15-30 psi (1.0-2.0 bar)) as the temperature within the autoclave may reach 121° C. (250° F.). Without the vented cap of the present disclosure, the short tube or pigtail in many instances collapses under the raised external pressure, wherein the air inside the short tube or pigtail is pushed into the medical fluid container or bag. Heating the thermoplastic tube or pigtail to the sterilization temperature causes it to become somewhat tacky. The collapsed tube thereby tends to stick closed to itself even after removal from the autoclave and cooling. At the time of use, the collapsed tube may become an impediment to good medical fluid, e.g., PD fluid, flow from the medical fluid container or bag to a desired treatment destination, e.g., a PD machine or cycler or to the patient's peritoneal cavity for continuous ambulatory peritoneal dialysis (“CAPD”).

It is accordingly expressly contemplated to provide an improved method for steam sterilizing medical fluid containers, such as PD fluid bags, in which the vented medical fluid supply line cap allows pressurized air from the surrounding autoclave atmosphere to enter the cap and the small tube or pigtail. The pressurized air entering the small tube or pigtail equalizes the pressure on either side of the tube, preventing the tube from collapsing. Also, the air heated to the sterilization temperature, e.g., 121° C. (250° F.), entering the small tube and a portion of the container or bag through the hydrophobic filter or vent brings the sterilization temperature directly to the surfaces contacted (including a portion of the medical fluid held within the container), such that the heat does not have to conduct through the tube and container or bag walls. In this manner, sterilization and sterilization efficiency are improved.

In light of the disclosure set forth herein, and without limiting the disclosure in any way, in a first aspect, which may be combined with any other aspect described herein, or portion thereof, a medical fluid container tubing assembly includes a medical fluid container, a luer connector including a luer port, a tube extending from the medical fluid container and terminating at the luer connector, and a cap fitted onto the luer connector. The cap includes a port sized to provide an interference fit with the luer port of the luer connector. The cap further includes a hydrophobic filter positioned and arranged to allow air to aseptically enter the tube to equalize pressure inside and outside of the tube.

In a second aspect, which may be combined with any other aspect described herein, or portion thereof, the cap and the luer connector are configured such that the cap translates onto and off of the luer connector.

In a third aspect, which may be combined with any other aspect described herein, or portion thereof, the cap and the luer connector are configured such that the cap threads onto and off of the luer connector.

In a fourth aspect, which may be combined with any other aspect described herein, or portion thereof, the luer port is a male luer port, and an inner surface of the port of the cap is sized and shaped to provide an interference fit with an outer surface of the male luer port.

In a fifth aspect, which may be combined with any other aspect described herein, or portion thereof, the luer connector includes an outer shroud, and an outer surface of the port of the cap includes at least one rib sized to provide a second interference fit with an inner surface of the outer shroud of the luer connector.

In a sixth aspect, which may be combined with any other aspect described herein, or portion thereof, the luer connector includes a first outer shroud, the cap includes a second outer shroud, and the first and second outer shrouds are sized such that a clearance space exists between an outer surface of the first outer shroud and an inner surface of the second outer shroud.

In a seventh aspect, which may be combined with any other aspect described herein, or portion thereof, an outer surface of the second outer shroud includes at least one rib for grasping the cap to connect and remove the cap from the luer connector.

In an eighth aspect, which may be combined with any other aspect described herein, or portion thereof, the cap defines a lumen that communicates fluidly with the tube, and the hydrophobic filter covers and opening formed by the lumen.

In a ninth aspect, which may be combined with any other aspect described herein, or portion thereof, the cap and the luer connector are configured such that a maximum force needed to remove the cap from the luer connector is 37 Newtons.

In a tenth aspect, which may be combined with any other aspect described herein, or portion thereof, a medical fluid supply line cap includes a body and an inner port formed by the body. The inner port includes an inner surface tapered for forming an interference fit with an outer surface of a male luer port of a luer connector. The medical fluid supply line cap also includes an outer shroud formed by the body. The outer shroud is sized to fit around an outer shroud of the luer connector. The body defines a lumen extending from the inner port through an opposing end of the body from the inner port to form an opening. The medical fluid supply line cap further includes a hydrophobic filter covering the opening formed by the lumen.

In an eleventh aspect, which may be combined with any other aspect described herein, or portion thereof, the hydrophobic filter is heat sealed, ultrasonically sealed, or solvent bonded to the body.

In a twelfth aspect, which may be combined with any other aspect described herein, the body defines a cavity, and the hydrophobic filter is located within the cavity to cover the opening formed by the lumen.

In a thirteenth aspect, which may be combined with any other aspect described herein, the cavity is cylindrical and the hydrophobic filter is circular.

In a fourteenth aspect, which may be combined with any other aspect described herein, at least one of an outer surface of the inner port or an outer surface of the shroud includes at least one rib extending longitudinally along the at least one outer surface.

In a fifteenth aspect, which may be combined with any other aspect described herein, an outer surface of the inner port is provided with male threads for threadingly engaging female threads provided by the luer connector.

In a sixteenth aspect, which may be combined with any other aspect described herein, a method for steam sterilizing a medical fluid container tubing assembly includes configuring a cap of the medical fluid container assembly to (i) form an interference fit with a luer connector and (ii) include a hydrophobic filter, which is positioned and arranged such that pressurized air aseptically enters the medical fluid container tubing assembly. The method also includes steam sterilizing the medical fluid container tubing assembly and a medical fluid container connected to the medical fluid container tubing assembly. Air heated to a sterilization temperature aseptically enters the medical fluid container tubing assembly through the hydrophobic filter and contacts an inside of at least a portion of the medical fluid container tubing assembly in addition to contacting an outside of the medical fluid container tubing assembly.

In a seventeenth aspect, which may be combined with any other aspect described herein, a tube is connected to the male luer connector, and the air heated to the sterilization temperature contacts an inside of at least a portion of at least one of the male luer connector or the tube.

In an eighteenth aspect, which may be combined with any other aspect described herein, the tube is connected to a medical fluid supply container, and the air heated to the sterilization temperature contacts a portion of the medical fluid within the supply container.

In a nineteenth aspect, which may be combined with any other aspect described herein, the air heated to the sterilization temperature equalizes pressure inside and outside of the tube.

In a twentieth aspect, any of the features, functionality and alternatives described in connection with any one or more of FIGS. 1 to 8 may be combined with any of the features, functionality and alternatives described in connection with any other of FIGS. 1 to 8.

In light of the above aspects and the present disclosure set forth herein, it is accordingly an advantage of the present disclosure to provide a medical fluid supply line cap that is vented.

It is another advantage of the present disclosure to provide a medical fluid supply line cap that prevents the line from collapsing during steam sterilization.

It is a further advantage of the present disclosure to provide a medical fluid supply line cap that may be used with many different medical fluids.

It is yet another advantage of the present disclosure to provide an improved method for steam sterilizing medical fluid containers, such as PD fluid supply bags.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a sectioned elevation section view of one embodiment of a medical fluid container tubing assembly of the present disclosure including a container tube, mating male luer connector and a vented medical fluid supply line cap.

FIG. 2 is a perspective view of one embodiment of the medical fluid container tubing assembly of the present disclosure including the container tube, mating male luer connector and vented medical fluid supply line cap.

FIG. 3 is a perspective view of one embodiment of the vented medical fluid supply line cap of the present disclosure.

FIG. 4 is a sectioned perspective view of one embodiment of the vented medical fluid supply line cap of the present disclosure.

FIG. 5 is a sectioned elevation view of one embodiment of the vented medical fluid supply line cap just prior to being applied to the mating male luer connector.

FIG. 6 is a sectioned elevation view of one embodiment of the vented medical fluid supply line cap applied to the mating male luer connector.

FIG. 7 is a sectioned elevation view of detail A of FIG. 6.

FIG. 8 is a sectioned elevation view of detail B of FIG. 6.

### DETAILED DESCRIPTION

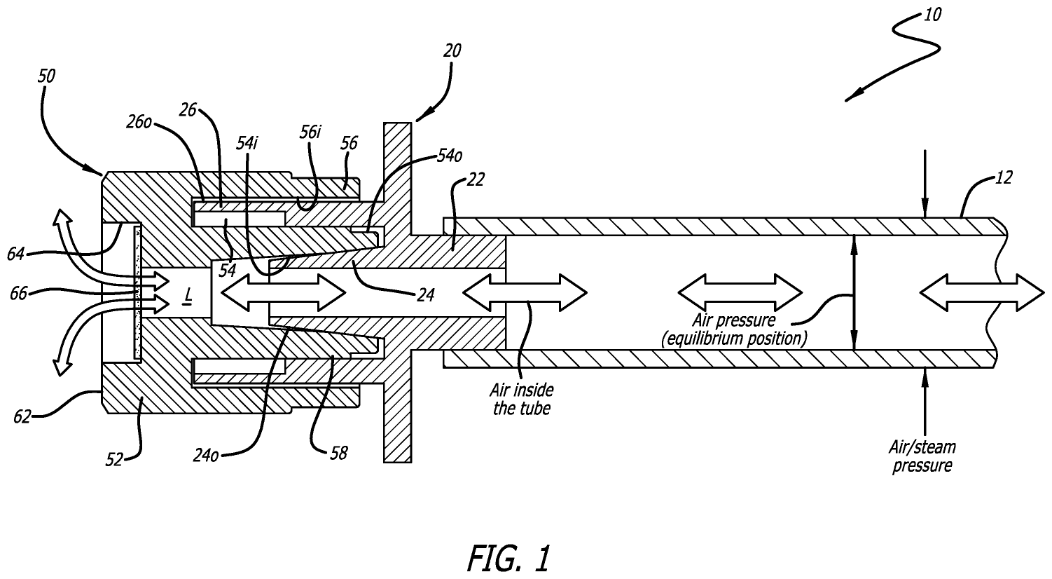

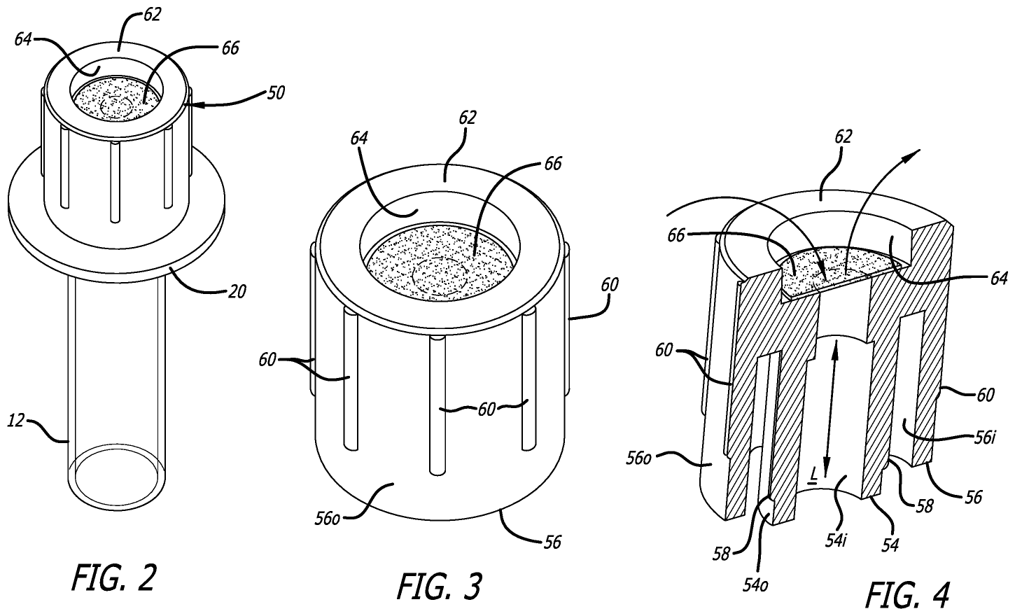

Referring now to the drawings and in particular to FIGS. 1 and 2, an embodiment of a medical fluid container tubing assembly 10 of the present disclosure is illustrated, and which includes a container tube 12, a mating male luer connector 20 and a vented medical fluid supply line cap 50. Container tube 12 extends from a medical fluid container or bag (not illustrated), which is considered as part of medical fluid container tubing assembly 10. The solution container or bag that is operable with any type of dialysis treatment using bagged dialysis fluid, including any type of peritoneal dialysis (“PD”) treatment, hemodialysis (“HD”) treatment, hemofiltration (“HF”) treatment, hemodiafiltration (“HDF”) treatment, or continuous renal replacement therapy (“CRRT”) treatment. It should be appreciated that vented medical fluid supply line cap 50 may be used in any type of medical treatment having a bagged or otherwise stored medical fluid, which needs to be opened aseptically for use, and which is steam sterilized. Vented medical fluid supply line cap 50 may therefore be used additionally with any type of bagged medical infusion or intravenous fluid, saline, lactated ringers, etc.

Vented medical fluid supply line cap 50 is in one embodiment configured to cap a luer connector, such as male luer connector 20. It should be appreciated however that medical fluid supply line cap 50 does not have to cap a luer connector and may instead cap a different type of connector, such as a connector that is spiked to allow medical fluid flow or a connector having a spike that spikes a mating connector to allow medical fluid flow. In any case, cap 50 in the illustrated embodiment caps a short line or tube 12, which may also be called a pigtail, wherein the short line or tube extends from the medical fluid supply container, e.g., a flexible bag, for a distance of less than one meter. The flexible bag may be a single chamber bag holding a fully mixed medical fluid or be a multi-chamber bag having one or more peel seal(s) separating medical fluid components that need to be isolated until the time of use. Short line or tube 12 may be heat sealed, ultrasonically sealed or solvent bonded to male luer connector 20.

Vented medical fluid supply line cap 50 is in one embodiment formed, e.g., molded as one piece, from a polymer, such as, polyetherimide (“PEI”), polyethersulfone (“PES”), polyamide/nylon (“PA”), acrylonitrile butadiene styrene (“ABS”), polycarbonate (“PC”) polyvinylchloride (“PVC”), nylon, polyether ether ketone (“PEEK”) and/or a thermoplastic elastomer, such as one marketed under the tradename Hytrel®. The medical fluid container or bag and short tube or pigtail 12 may be made of PVC or other suitable medically safe material.

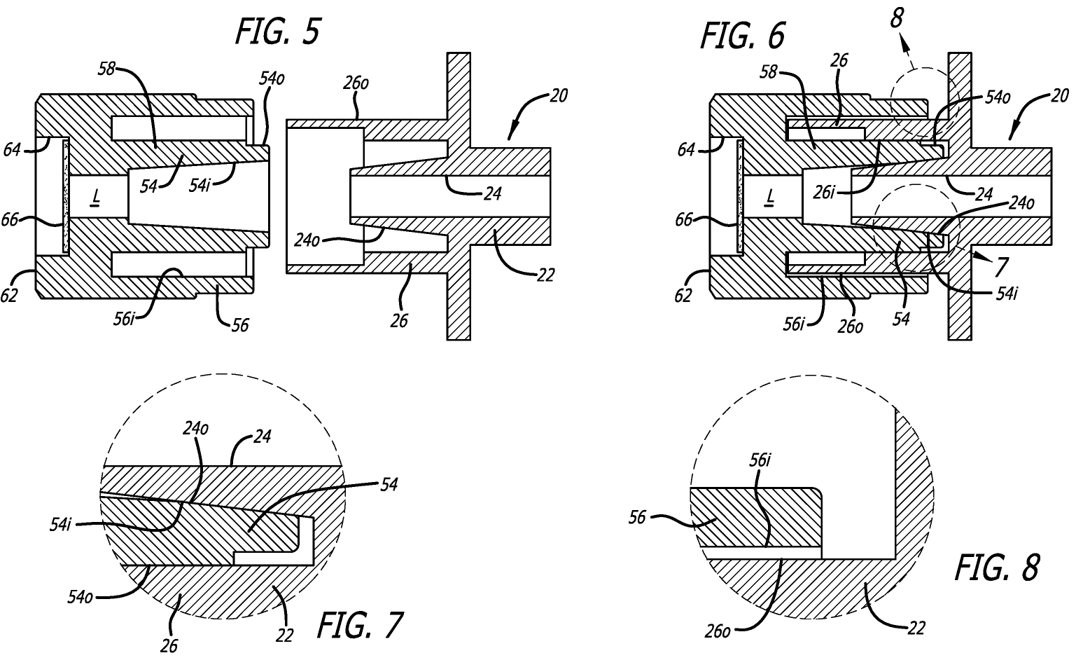

FIGS. 1, 5, 6 and 7 illustrate that where vented medical fluid supply line cap 50 is configured to cap a luer connector, the cap is one embodiment configured to have female luer features, while mating luer connector 20 is a male luer connector. In alternative embodiments, cap 50 may be provided with male luer features, while mating luer connector 20 is a female luer connector. Where the vented medical fluid supply line cap is configured as having female luer features as illustrated in FIGS. 1, 5, 6 and 7, cap 50 includes a body 52 having or defining an inner port 54 and an outer shroud 56. Mating luer connector 20 in turn includes a body 22 having or defining an inner male luer port 24 and an outer shroud 26.

FIGS. 6 and 7 highlight that an inner surface 54i of inner port 54 in one embodiment includes or defines a female luer taper (e.g., a five to ten degree taper), which forms an interference fit with an outer surface 24o of inner male luer port 24 of mating male luer connector 20. FIGS. 4, 5 and 6 highlight that an outer surface 54o of inner port 54 is in one embodiment provided with a plurality of ribs 58, e.g., extending longitudinally along outer wall 54o. Ribs 58 form a secondary interference fit with female threads provided on an inner surface 26i of outer shroud 26 of mating male luer connector 20. The female threads threadingly connect to male threads of a female luer connector (not illustrated) to establish medical fluid flow after vented medical fluid supply line cap 50 has been removed from male luer connector 20.

FIGS. 1, 6 and 8 highlight that an inner surface 56i of outer shroud 56 of vented medical fluid supply line cap 50 is sized in one embodiment to provide a slight amount of clearance space with an outer surface 26o of outer shroud 26 of mating male luer connector 20. The clearance space allows the interference fit between inner surface 54i of inner port 54 of cap 50 and outer surface 24o of inner male luer port 24 of mating male luer connector 20 to be the primary interference fit that prevents medical fluid from leaking out of vented medical fluid supply line cap 50 when placed or fitted onto mating male luer connector 20.

In one embodiment, vented medical fluid supply line cap 50 is translated onto and off of mating male luer connector 20 with the interference fits described above holding the cap onto the male luer connector. Table 1 below shows that in one embodiment a maximum translational removal force is set to 37 Newtons (“N”). Five different tests each confirmed that the configuration of vented medical fluid supply line cap 50 illustrated herein met the force removal goal:

TABLE 1

| | | | Pull |
|---|---|---|---|
| | | | Force |
| | | Sample | (N) |
| Pull force test of the cap | vented medical fluid | sample#1 | 27.3 |
| from Luer (pull force | supply line cap 50 shall | sample#2 | 31.4 |
| measured by tensile testing | have a maximum | sample#3 | 33.0 |
| equipment with load | removal force from | sample#4 | 26.7 |
| speed = 400 mm/min) | mating male luer | sample#5 | 35.8 |
| | connector 20 of 37N. | | |

In an alternative embodiment, the plurality of ribs 58 provided on outer surface 54o of inner port 54 of cap 50 are replaced with male luer threads that threadingly connect with the female luer threads provided on inner surface 26i of outer shroud 26 of mating male luer connector 20. Here, cap 50 threads onto (making the primary interference fit) and off of the mating male luer connector 20 in the same manner as the female luer connector (not illustrated) uses to establish medical fluid flow. In either situation (translated or threaded), FIGS. 3 and 4 illustrate that an outer surface 56o of outer shroud 56 of cap 50 is formed with a plurality of ribs 60, e.g., longitudinally extending ribs, which aid the user in translating or threading cap 50 onto or off of the mating male luer connector 20.

FIGS. 1, 4, 5 and 6 illustrate that in an embodiment, a lumen L formed by inner surface 54i of inner port 54 of vented medical fluid supply line cap 50 extends through body 52, to and through a circular distal end portion 62 of the body. FIGS. 1 to 6 illustrate that an enlarged diameter cavity 64 is provided at the distal end of the body in one embodiment. Cavity 64 provides a location to attach a vent 66, such as a hydrophobic membrane filter, within cavity 64, such that the opening or lumen L through body 52 of cap 50 is covered. In the illustrated embodiment, cavity 64 is cylindrically shaped while hydrophobic filter or vent 66 is circular. Hydrophobic filter or vent 66 allows air but not medical fluid to flow through the filter or vent, into or out of body 52 of cap 50. Also, air flowing into body 52 of cap 50 is aseptically filtered via hydrophobic filter or vent 66. In an embodiment, hydrophobic filter or vent 66 includes a 0.2 micron polytetrafluoroethylene (“PTFE”) hydrophobic membrane having a polyester substrate. Hydrophobic filter or vent 66 may be sealed around its outer diameter to cavity 64 of body 52 via heat sealing, ultrasonic sealing or solvent bonding.

When a medical fluid container or bag filled with medical fluid and having a short tube or pigtail 12 extending from the container, which is capped by vented cap 50 of the present disclosure, is steam sterilized, hydrophobic filter or vent 66 prevents short tube or pigtail 12 from collapsing. Steam sterilization typically involves many medical fluid containers or bags being placed within an autoclave and steam sterilized at the same time. The external environment around the medical fluid container or bag is filled with steam, raising the pressure in the autoclave to above ambient pressure (e.g., 15-30 psi (1.0-2.0 bar)) as the temperature within the autoclave may reach 121° C. (250° F.). Without vented cap 50 of the present disclosure, short tube or pigtail 12 in many instances collapses under the raised external pressure, wherein the air inside short tube or pigtail 12 is pushed into the medical fluid container or bag. Heating the thermoplastic tube or pigtail 12 to the sterilization temperature causes it to become somewhat tacky. The collapsed tube thereby tends to stick closed to itself even after removal from the autoclave and cooling. At the time of use, the collapsed tube may become an impediment to good medical fluid, e.g., PD fluid, flow from the medical fluid container or bag to a desired treatment destination, e.g., a PD machine or cycler or to the patient's peritoneal cavity for continuous ambulatory peritoneal dialysis (“CAPD”).

The airflow arrows of FIGS. 1 and 4 illustrate that it is expressly contemplated to provide an improved method for steam sterilizing medical fluid containers, such as PD fluid bags, in which vented medical fluid supply line cap 50 allows pressurized air from the surrounding autoclave atmosphere to enter the cap and small tube or pigtail 12. Pressurized air entering small tube or pigtail 12 equalizes the pressure on either side of the tube, preventing the tube from collapsing. Also, the air heated to the sterilization temperature, e.g., 121° C. (250° F.), entering small tube 12 and a portion of the container or bag through hydrophobic filter or vent 66 brings the sterilization temperature directly to the inner surfaces of medical fluid container tubing assembly 10, including tube 12 and possibly even to a portion of the medical fluid held within the container, such that the heat does not have to conduct through the tube 12 and container or bag walls. In this manner, heat sterilization and heat sterilization efficiency are improved.

It should be understood that various changes and modifications to the presently preferred embodiments described herein will be apparent to those skilled in the art. It is therefore intended that any or all of such changes and modifications may be covered by the appended claims.

The invention is claimed as follows:

1. A medical fluid container tubing assembly comprising:
a medical fluid container;
a luer connector including a luer port;
a tube extending from the medical fluid container and terminating at the luer connector; and
a cap fitted onto the luer connector, the cap including a port sized to provide an interference fit with the luer port of the luer connector, the cap further including a hydrophobic filter positioned and arranged to allow air to aseptically enter the tube to equalize pressure inside and outside of the tube.

2. The medical fluid container tubing assembly of claim 1, wherein the cap and the luer connector are configured such that the cap translates onto and off of the luer connector.

3. The medical fluid container tubing assembly of claim 1, wherein the cap and the luer connector are configured such that the cap threads onto and off of the luer connector.

4. The medical fluid container tubing assembly of claim 1, wherein the luer port is a male luer port, and wherein an inner surface of the port of the cap is sized and shaped to provide an interference fit with an outer surface of the male luer port.

5. The medical fluid container tubing assembly of claim 1, wherein the luer connector includes an outer shroud, and wherein an outer surface of the port of the cap includes at least one rib sized to provide a second interference fit with an inner surface of the outer shroud of the luer connector.

6. The medical fluid container tubing assembly of claim 1, wherein the luer connector includes a first outer shroud, the cap includes a second outer shroud, and wherein the first and second outer shrouds are sized such that a clearance space exists between an outer surface of the first outer shroud and an inner surface of the second outer shroud.

7. The medical fluid container tubing assembly of claim 6, wherein an outer surface of the second outer shroud includes at least one rib for grasping the cap to connect and remove the cap from the luer connector.

8. The medical fluid container tubing assembly of claim 1, wherein the cap defines a lumen that communicates fluidly with the tube, and wherein the hydrophobic filter covers and opening formed by the lumen.

9. The medical fluid container tubing assembly of claim 1, wherein the cap and the luer connector are configured such that a maximum force needed to remove the cap from the luer connector is 37 Newtons.

10. A medical fluid supply line cap comprising:
a body;
an inner port formed by the body, the inner port including an inner surface tapered for forming an interference fit with an outer surface of a male luer port of a luer connector;
an outer shroud formed by the body, the outer shroud sized to fit around an outer shroud of the luer connector;
a lumen defined by the body that extends from the inner port through an opposing end of the body from the inner port to form an opening; and
a hydrophobic filter covering the opening formed by the lumen.

11. The medical fluid supply line cap of claim 10, wherein the hydrophobic filter is heat sealed, ultrasonically sealed, or solvent bonded to the body.

12. The medical fluid supply line cap of claim 10, wherein the body defines a cavity, and wherein the hydrophobic filter is located within the cavity to cover the opening formed by the lumen.

13. The medical fluid supply line cap of claim 12, wherein the cavity is cylindrical and the hydrophobic filter is circular.

14. The medical fluid supply line cap of claim 12, wherein at least one of an outer surface of the inner port or an outer surface of the shroud includes at least one rib extending longitudinally along the at least one outer surface.

15. The medical fluid supply line cap of claim 12, wherein an outer surface of the inner port is provided with male threads for threadingly engaging female threads provided by the luer connector.

16. A method for steam sterilizing a medical fluid container tubing assembly, the method comprising:
configuring a cap of the medical fluid container assembly to (i) form an interference fit with a luer connector and (ii) include a hydrophobic filter, the hydrophobic filter positioned and arranged such that pressurized air aseptically enters the medical fluid container tubing assembly; and
steam sterilizing the medical fluid container tubing assembly and a medical fluid container connected to the medical fluid container tubing assembly, wherein air heated to a sterilization temperature aseptically enters the medical fluid container tubing assembly through the hydrophobic filter and contacts an inside of at least a portion of the medical fluid container tubing assembly in addition to contacting an outside of the medical fluid container tubing assembly.

17. The steam sterilization method of claim 16, wherein a tube is connected to the male luer connector, and wherein the air heated to the sterilization temperature contacts an inside of at least a portion of at least one of the male luer connector or the tube.

18. The steam sterilization method of claim 17, wherein the tube is connected to a medical fluid supply container, and wherein the air heated to the sterilization temperature contacts a portion of the medical fluid within the supply container.

19. The steam sterilization method of claim 17, wherein the air heated to the sterilization temperature equalizes pressure inside and outside of the tube.
